(19) United States
(12) Patent Application Publication
(10) Pub. No.: US 2026/0171221 A1
(43) Pub. Date: Jun. 18, 2026

(54) APPARATUS AND METHOD FOR MEASURING BIOSIGNAL BASED ON THE rPPG TECHNOLOGY AND MOST FREQUENT INTERVAL

(71) Applicant: EMMA HEALTHCARE CO., LTD., Seongnam-si (KR)

(72) Inventor: Ryanghee SOHN, Seongnam-si (KR)

(73) Assignee: EMMA HEALTHCARE CO., LTD., Seongnam-si (KR)

(21) Appl. No.: 19/418,460

(22) Filed: Dec. 12, 2025

(30) Foreign Application Priority Data
Dec. 18, 2024 (KR) 10-2024-0189928

(51) Int. Cl. G16H 30/40; A61B 5/00; G06V 10/56; G06V 40/16

(52) U.S. Cl. CPC G16H 30/40; A61B 5/0064; G06V 10/56; G06V 40/162; G06V 40/171

(57) ABSTRACT

The apparatus comprises a camera configured to obtain an image including a face of user; and a processor operably coupled to the camera, and configured to: recognize a facial region of the user from the obtained image and divide the facial region into a plurality of sub-regions according to a predetermined method; convert each of the sub-regions into a predetermined color coordinate system and calculate, for each of the sub-regions, an average value of a color component included in the predetermined color coordinate system; extract, among predefined intervals, a most frequent interval corresponding to an interval in which the average values of the color component calculated for the respective sub-regions are most frequently distributed; extract a central interval among a predetermined number of recently extracted most frequent intervals; and extract an image including the sub-regions whose calculated average values of the color component correspond to the extracted central interval.

### BACKGROUND OF THE INVENTION

### Field of the Invention

This application claims the benefit of Korean Patent Application No. 10-2024-0189928, filed on Dec. 18, 2024, which is hereby incorporated by reference as if fully set forth herein. The present invention relates to an apparatus and a method for effectively measuring biosignals based on remote photoplethysmography (rPPG) technology. This invention was made with support from the Ministry of Trade, Industry and Energy (MOTIE), the Korea Institute for Advancement of Technology (KIAT), under the International Joint Technology Development Program for Industrial Technology Cooperation. The project is entitled “Development of AI-Based Emotion Evaluation Technology for Alzheimer's Patients Using Facial Images and EEG Signals,” Project No. P0026265, and was conducted during the period from Jan. 1, 2025 to Dec. 31, 2025.

### Description of the Related Art

In recent years, in the medical and healthcare fields, rPPG (remote Photoplethysmogram) technology—which measures a user's biosignals in a non-contact manner using a camera—has attracted significant attention. This technology analyzes subtle variations in skin color captured in camera images to measure various biosignals such as heart rate, respiratory rate, and blood oxygen saturation. Accordingly, its use is expected to expand across a wide range of applications, including patient monitoring in medical institutions and daily health-management scenarios.

However, conventional rPPG technologies face several technical limitations due to the requirement of accurately detecting minute changes in skin color. In particular, measurement values can be severely distorted when lighting conditions change in indoor or outdoor environments or when the user's face moves. This occurs because the intensity of light reflected from the skin varies depending on ambient lighting conditions, and facial movement makes it difficult to continuously track specific skin regions. These constraints make it extremely difficult to obtain reliable measurements in real-world environments.

### SUMMARY OF THE INVENTION

It is an object of the present invention to provide an apparatus and a method for measuring biosignals based on rPPG.

In accordance with an aspect of the present invention, the above and other objects can be accomplished by the provision of an apparatus for measuring a biosignal based on remote photoplethysmography (rPPG) comprises a camera configured to obtain an image including a face of a user; and a processor operably coupled to the camera, and configured to: recognize a facial region of the user from the obtained image and divide the facial region into a plurality of sub-regions according to a predetermined method; convert each of the sub-regions into a predetermined color coordinate system and calculate, for each of the sub-regions, an average value of a color component included in the predetermined color coordinate system; extract, among predefined intervals, a most frequent interval corresponding to an interval in which the average values of the color component calculated for the respective sub-regions are most frequently distributed; when a difference between the extracted most frequent interval and a most frequent interval extracted immediately prior thereto is greater than or equal to a predetermined threshold, extract a central interval among a predetermined number of recently extracted most frequent intervals; and extract an image including the sub-regions whose calculated average values of the color component correspond to the extracted central interval.

When the central interval among the predetermined number of recently extracted most frequent intervals comprises two intervals, the processor is further configured to calculate central values of the two intervals and extract, as the central interval, a single interval that includes an average of the calculated central values.

The processor is further configured to output an RGB signal corresponding to variations of R (Red), G (Green), and B (Blue) based on the image for the sub-regions corresponding to the most frequent interval.

The processor is configured to provide the output RGB signal to a Photoplethysmography (PPG) signal-processing device, an rPPG signal-processing device, or a trained artificial-intelligence model to measure various biosignals.

The processor is further configured to calculate an average RGB value of an image including the sub-regions whose calculated average values of the color component correspond to the extracted central interval.

The color coordinate system is any one of RGB, HSV, CIELab, YCbCr, Grayscale, or YUV.

The color component is: any one of R (Red), G (Green), or B (Blue) when the color coordinate system is RGB; any one of H (Hue), S (Saturation), or V (Value) when the color coordinate system is HSV; any one of L (Lightness), a (a degree ranging from red(+) to green(−)), or b (a degree ranging from yellow(+) to blue(−)) when the color-coordinate system is CIELab; any one of Y (luminance), Cb (blue-difference chroma signal), or Cr (red-difference chroma signal) when the color-coordinate system is YCbCr; a grayscale brightness value when the color-coordinate system is Grayscale; and any one of Y (luminance), U (blue luminance), or V (red luminance) when the color-coordinate system is YUV.

The processor is configured to recognize the facial region of the user from the obtained image and divide the facial region into a plurality of sub-regions by using a Mediapipe.

In accordance with an aspect of the present invention, the above and other objects can be accomplished by the provision of method for measuring a biosignal based on remote photoplethysmography (rPPG) comprises obtaining an image including a face of a user; recognizing a facial region of the user from the obtained image and dividing the facial region into a plurality of sub-regions according to a predetermined method; converting each of the sub-regions into a predetermined color coordinate system and calculate, for each of the sub-regions, an average value of a color component included in the predetermined color coordinate system; extracting, among predefined intervals, a most frequent interval corresponding to an interval in which the average values of the color component calculated for the respective sub-regions are most frequently distributed; when a difference between the extracted most frequent interval and a most frequent interval extracted immediately prior thereto is greater than or equal to a predetermined threshold, extracting a central interval among a predetermined number of recently extracted most frequent intervals; and extracting an image including the sub-regions whose calculated average values of the color component correspond to the extracted central interval.

The method further comprises, wherein when the central interval among the predetermined number of recently extracted most frequent intervals comprises two intervals, calculating central values of the two intervals and extract, as the central interval, a single interval that includes an average of the calculated central values.

The method further comprises calculating an average RGB value of an image including the sub-regions whose calculated average values of the color component correspond to the extracted central interval.

In accordance with an aspect of the present invention, the above and other objects can be accomplished by the provision of non-transitory computer-readable storage medium storing instructions that, when executed by one or more processors, cause the one or more processors to: obtain an image including a face of a user; recognize a facial region of the user from the obtained image and divide the facial region into a plurality of sub-regions according to a predetermined method; convert each of the sub-regions into a predetermined color coordinate system and calculate, for each of the sub-regions, an average value of a color component included in the predetermined color coordinate system; extract, among predefined intervals, a most frequent interval corresponding to an interval in which the average values of the color component calculated for the respective sub-regions are most frequently distributed; when a difference between the extracted most frequent interval and a most frequent interval extracted immediately prior thereto is greater than or equal to a predetermined threshold, extracting a central interval among a predetermined number of recently extracted most frequent intervals; and extract an image including the sub-regions whose calculated average values of the color component correspond to the extracted central interval.

### BRIEF DESCRIPTION OF THE DRAWINGS

The accompanying drawings, which are incorporated in and constitute a part of the detailed description, provide exemplary embodiments of the present invention and, together with the detailed description, serve to explain the technical spirit of the invention.

FIG. 1 is an exemplary diagram illustrating rPPG.

FIG. 2 is a block diagram illustrating the functions of the apparatus 300 for measuring a biosignal based on an rPPG according to the present invention.

FIG. 3 is an exemplary diagram illustrating the facial region of the user divided into the plurality of sub-regions by the apparatus 300 for measuring a biosignal based rPPG according to the present invention.

FIG. 4 is an exemplary diagram illustrating a portion of the data-processing procedure of the apparatus 300 for measuring a biosignal based rPPG according to the present invention.

FIG. 5 is an exemplary diagram illustrating extracting a most frequent interval by the apparatus 300 for measuring biosignal based on the rPPG technology according to the present invention.

FIG. 6 is another exemplary diagram for explaining a part of the data-processing procedure of the apparatus 300 according to the present invention.

FIG. 7 is an exemplary diagram illustrating a process of extracting a central interval in the apparatus 300 according to the present invention.

### DETAILED DESCRIPTION OF THE INVENTION

The present invention may be embodied in various forms and may have multiple embodiments. Specific embodiments are illustrated in the drawings and will be described in detail; however, these are presented merely to exemplify the invention, and are not intended to limit the invention to particular embodiments. It should be understood that all modifications, equivalents, and alternatives falling within the spirit and scope of the invention are intended to be encompassed.

When an element is described as being ‘connected to’ or ‘coupled to’ another element, the element may be directly connected or coupled to the other element, or other intervening elements may be present. In contrast, when an element is described as being ‘directly connected to’ or ‘directly coupled to’ another element, no intervening elements are present.

The terms ‘first,’ ‘second,’ and the like may be used to describe various elements, but such terms shall not limit the elements by their numerical designation. These terms are used merely to distinguish one element from another.

The terminology used herein is for the purpose of describing particular embodiments only and is not intended to limit the invention. As used herein, the singular forms include the plural forms unless the context clearly indicates otherwise. The terms ‘include’ and ‘have,’ and any variations thereof, are intended to specify the presence of stated features, integers, steps, operations, components, or combinations thereof, but do not preclude the presence or addition of one or more other features, integers, steps, operations, components, or combinations thereof.

Further, the terms ‘unit,’ ‘module,’ ‘part,’ or ‘device,’ as used herein, refer to a functional element configured to perform at least one operation or function, and may be implemented in hardware, software, or a combination thereof.

In some cases, well-known structures and devices may be omitted or shown in block diagram form to avoid obscuring the concept of the invention. Throughout the drawings, identical reference numerals denote identical or similar components.

The components described in connection with the embodiments with reference to the drawings are not necessarily limited to the specific embodiments, and other embodiments may include the components so long as the technical spirit of the invention is maintained. Multiple embodiments may also be combined into a single embodiment, even if not expressly described.

Before describing the invention in detail, the concepts of color coordinate systems and color components are introduced.

A color coordinate system is a mathematical model for representing colors in a digital image. Each color coordinate system has its own characteristics, advantages, and disadvantages, and is sometimes referred to as a color space. In this specification, the term color-coordinate system is used.

Color coordinate systems have evolved based on human visual perception, display characteristics, or requirements of particular applications. In image processing and computer vision, selecting an appropriate color-coordinate system according to the purpose of the task is crucial, and conversions between color-coordinate systems allow desired characteristics to be effectively utilized.

A color component refers to the individual numerical value representing a specific aspect of color within a color coordinate system. Color component is also called color channel or color space element. In this specification, the term ‘color component’ is used.

Each color component has a defined meaning and numeric range within the corresponding color coordinate system, and the combination of color components allows representation of a wide variety of colors.

The RGB color coordinate system is the most basic and widely used system, consisting of three primary color components: R (Red), G (Green), and B (Blue). Each component typically ranges from 0 to 255, where (0,0,0) represents black and (255,255,255) represents white. RGB aligns well with display device characteristics and is intuitive and easy to implement in hardware, but it may not fully reflect human color perception and may have limitations in color editing or analysis.

The HSV color coordinate system is described as follows.

The HSV color coordinate system consists of three components: Hue (H), Saturation (S), and Value (V), and when visualized, it forms a three-dimensional conical structure. Hue (H) is represented as an angle from 0° to 360° and denotes the base color. Saturation (S) represents the purity of the color with a value ranging from 0 to 1, and Value (V) denotes brightness, also ranging from 0 to 1. Because HSV is similar to human color perception, it is highly useful for color selection and editing tasks. It is particularly effective in computer-vision applications for color-based object detection and tracking.

The CIELab color coordinate system is a standard color model proposed by the International Commission on Illumination (CIE) and is designed to reflect human visual perception characteristics. It consists of three components: L (Lightness), a, and b. The L component ranges from 0 (black) to 100 (white), the a component represents the degree from red(+) to green(−), and the b component represents the degree from yellow(+) to blue(−). The CIELab model provides perceptual uniformity, allowing color differences to be represented numerically with high accuracy.

The YCbCr color-coordinate system is widely used in digital image compression and transmission and consists of Y (luminance/brightness), Cb, and Cr. The Y component represents luminance, while Cb and Cr represent chrominance information corresponding to the difference between the blue component and luminance, and the difference between the red component and luminance, respectively. By exploiting the fact that human vision is more sensitive to luminance than to chrominance, YCbCr enables efficient data compression by applying greater compression to the chrominance signals. For this reason, it is extensively used in digital video compression standards such as JPEG and MPEG.

The Grayscale color-coordinate system is the simplest form of color representation and includes only a brightness component. Brightness is expressed with a value from 0 (black) to 255 (white). A grayscale image is typically generated by computing a weighted average of the R, G, and B components of each pixel, commonly using the formula Y=0.299R+0.587G+0.114B, which reflects the characteristic that human vision is most sensitive to green and least sensitive to blue.

The YUV color-coordinate system was developed for analog color television broadcasting and consists of Y (luminance/brightness), U (blue-luminance chrominance), and V (red-luminance chrominance). Although YUV operates on principles similar to YCbCr, it employs scaling and offsets tailored for analog signal transmission. The YUV model played an essential role in maintaining compatibility between color TV signals and monochrome TV receivers in analog standards such as NTSC and PAL.

Conversions between color coordinate systems play a crucial role in image processing. For example, an RGB image captured by a camera may be converted into HSV for color analysis or into YCbCr for efficient compression. Each conversion follows a specific mathematical formula, and some conversions may involve information loss; thus, the selection of an appropriate color-coordinate system depends on the intended application.

To convert an RGB image into HSV, Equation 1 below may be used

$${R}^{′} = R / 255 \qquad [ Equation 1 ]$$

$${G}^{′} = G / 255$$

$${B}^{′} = B / 255$$

$${C}_{max} = max ( {R}^{′} , {G}^{′} , {B}^{′} )$$

$${C}_{min} = min ( {R}^{′} , {G}^{′} , {B}^{′} )$$

$$Δ = {C}_{max} / {C}_{min}$$

$$H = \{ \begin{matrix}0 ° , & Δ = 0 \\ 60 ° \times ( \frac{{G}^{′} - {B}^{′}}{Δ} mod 6 ) , & {C}_{max} = {R}^{′} \\ 60 ° \times ( \frac{{B}^{′} - {R}^{′}}{Δ} + 2 ) , & {C}_{max} = {G}^{′} \\ 60 ° \times ( \frac{{R}^{′} - {G}^{′}}{Δ} + 4 ) , & {C}_{max} = {B}^{′}\end{matrix}$$

$$S = \{ \begin{matrix}0 ° , & {C}_{max} = 0 \\ \frac{Δ}{{C}_{max}} , & {C}_{max} \neq 0\end{matrix}$$

$$V = {C}_{max}$$

The remote photoplethysmogram (rPPG) technology is described as follows.

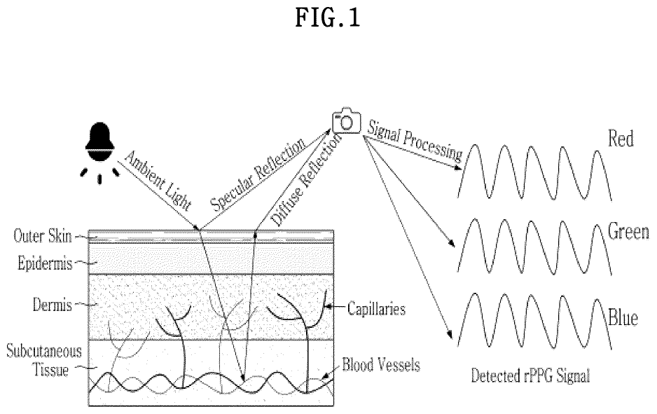

FIG. 1 is an exemplary diagram illustrating rPPG.

Various biosignals such as heart rate, heart-rate variability, and other physiological parameters can be measured remotely using PPG technology. As illustrated in FIG. 1, remote measurement of heart rate and the like can be performed by capturing high-resolution video with a camera. This technique may be useful for various physical, health-related, and emotional monitoring applications, including driver monitoring, elderly monitoring, and infant monitoring. Although rPPG operates based on the same principle as PPG, it performs contactless measurement. It measures variations in reflected red, green, and blue light from the skin, which arise from contrasts between specular reflection and diffuse reflection. Specular reflection corresponds to the direct reflection of light from the skin surface, whereas diffuse reflection corresponds to reflected light affected by absorption and scattering in skin tissues, which vary according to changes in blood volume. This principle utilizes the fact that hemoglobin reflects red light and absorbs green light.

FIG. 1 illustrates waveforms of red, green, and blue light detected after performing signal processing using a remote PPG technique, showing that the rPPG waveforms detected for each wavelength differ from one another. Remote measurement technology for biosignal monitoring is particularly useful in scenarios requiring non-contact monitoring, such as infant monitoring or infectious-disease situations like COVID-19, because it enables prediction of biosignals based solely on facial information without physical contact. Facial images are acquired using ambient light surrounding the subject as the illumination source.

As illustrated in FIG. 1, technologies utilizing rPPG generally employ temporal variations of the R (Red), G (Green), and B (Blue) color components of a region of interest as input signals. Each of the RGB-component signals may also be referred to as an rPPG signal and may be used to measure various biosignals through a PPG signal-processing device, an rPPG signal-processing device, or a trained artificial-intelligence model.

The present invention proposes an rPPG-based biosignal measurement device and method in which the face is segmented into key regions based on facial landmarks, and noise is effectively removed by utilizing the color components of selected color-coordinate systems to achieve optimized noise-reduction performance

The present invention proposes an innovative approach that leverages color coordinate systems to address these challenges.

Specifically, the color coordinate system recognizes the user's face and subdivides it into key regions based on facial landmarks, and applies optimized noise-removal techniques for each region. In particular, filtering algorithms are introduced based on the color composition of a selected color-coordinate system, tailored to the characteristics of each facial region, thereby enabling effective discrimination and removal of noise caused by lighting variations and movement.

Through this technology, the invention significantly overcomes the limitations of existing rPPG systems. Even under abrupt changes in lighting conditions or natural movements of the user, biosignals can be measured stably, greatly enhancing reliability and accuracy in real-world usage environments. This enables more practical and effective biosignal measurement across diverse applications, including patient monitoring in clinical settings and daily health-status assessment.

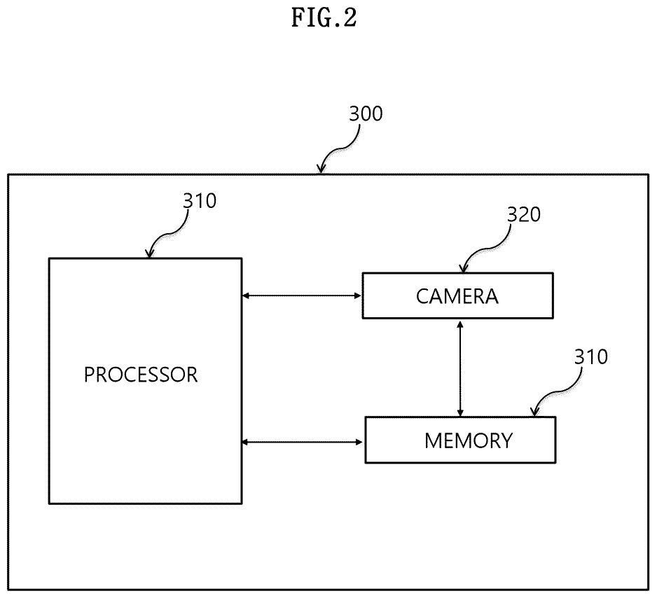

FIG. 2 is a block diagram illustrating the functions of the apparatus 300 for measuring a biosignal based on an rPPG according to the present invention.

Referring to FIG. 2, the apparatus 300 for measuring a biosignal based on an rPPG according to the present invention may include a processor 310, a camera 320, and a memory 330.

The camera 320 may obtain or acquire an image including a face of a user. The image including the user's face may be a single photograph, a sequence of photographs, or a video.

The processor 310 is operably and electrically coupled to the camera 320 and the memory 330 through one or more wired or wireless communication interfaces.

The processor 310 may recognize the facial region of the user and divide the facial region into a plurality of sub-regions according to a predetermined method. In particular, the processor 310 may use facial landmarks corresponding to major portions of the face in order to divide the facial region into the plurality of sub-regions, and the sub-regions may be formed in polygonal shapes including triangular regions.

In one example, the processor 310 may use the Facemesh function of Mediapipe distributed by Google to recognize the user's face and extract facial landmarks, and may divide the facial region into the plurality of sub-regions based on the extracted facial landmarks using Mediapipe's Facemesh.

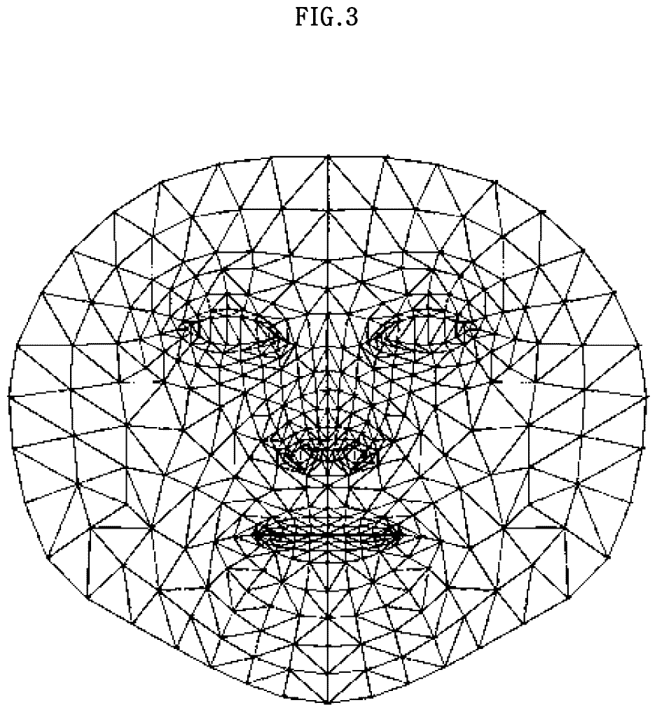

FIG. 3 is an exemplary diagram illustrating the facial region of the user divided into the plurality of sub-regions by the apparatus 300 for measuring a biosignal based rPPG according to the present invention.

Referring to FIG. 3, it can be seen that the processor 310 recognizes the user's face from the image including the user's face, extracts the facial landmarks, and divides the facial region into the plurality of sub-regions.

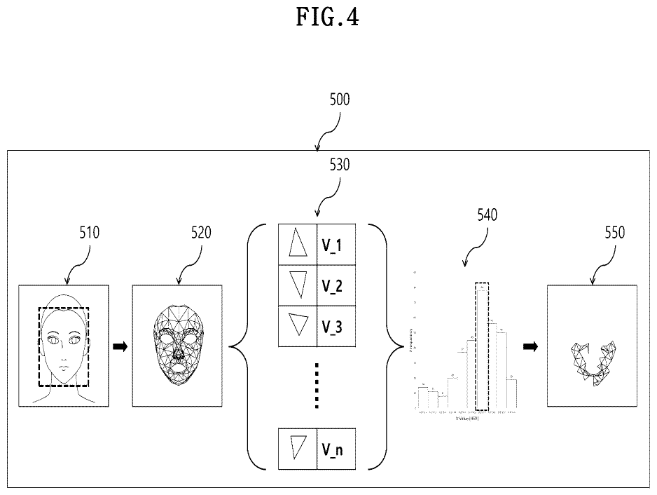

FIG. 4 is an exemplary diagram illustrating a portion of the data-processing procedure of the apparatus 300 for measuring a biosignal based rPPG according to the present invention.

Referring to FIG. 4, the processor 310 may perform a step 510 of recognizing a face of a user, and may perform a step 520 of dividing the facial region into a plurality of sub-regions based on facial landmarks corresponding to major portions of the recognized face.

For each of the divided sub-regions, the processor 310 may convert the sub-region into a predetermined color-coordinate system and calculate an average value of a color component (or color channel) included in the color-coordinate system. The color component corresponds to a color space element.

When the camera 320 outputs the obtained image in an RGB color coordinate system, and when the HSV color-coordinate system is used in subsequent data processing, the processor 310 may convert the RGB image data into the HSV color coordinate system by using Equation 1 described above.

Referring to FIG. 4, the processor 310 may perform a step 530 of converting each of the divided sub-regions into the HSV color coordinate system and calculating an average value of the V (Value, brightness) color component included in the HSV color-coordinate system. Here, the left side of the illustrated table represents the shape of each sub-region, and the right side of the table represents the corresponding average value of the V color component for that sub-region. That is, the first sub-region and its corresponding average V value are shown as V₁, the second sub-region and its corresponding average V value are shown as V₂, and the last sub-region and its corresponding average V value are shown as Vₙ.

The processor 310 may extract a most frequent interval corresponding to an interval in which a plurality of average values of the color component calculated for the respective sub-regions are most frequently distributed among predefined intervals.

Referring to FIG. 4, the processor 310 may perform a step 540 of extracting, based on the V color component of the HSV color coordinate system, a most frequent interval corresponding to the interval in which the average values of the V color component calculated for the respective sub-regions are most frequently distributed among the predefined intervals.

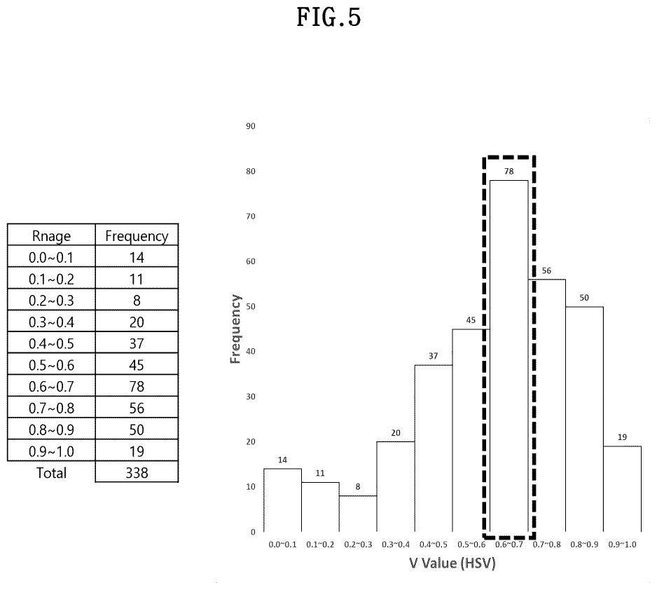

FIG. 5 is an exemplary diagram illustrating extracting a most frequent interval by the apparatus 300 for measuring biosignal based on the rPPG technology according to the present invention.

FIG. 5 illustrates, in greater detail, step 540 in which the processor extracts a most frequent interval corresponding to an interval in which the average values of the V (Value, brightness) component of the HSV color coordinate system, calculated for the respective sub-regions, are most frequently distributed among predefined intervals.

Referring to FIG. 5, the V component of the HSV color coordinate system is predefined in intervals of 0.1, and the processor 310 may determine the frequency of the average V values of the color component for the respective sub-regions within each predefined interval, and extract the interval having the highest frequency. In this example, the recognized facial region is divided into a total of 338 sub-regions, and the most frequent interval in which the average V values of the color component are most frequently distributed is the interval greater than 0.6 and less than or equal to 0.7 (0.6 to 0.7). A total of 78 sub-regions out of 338 fall within this interval. Thus, it can be seen from FIGS. 4 and 5 that the most frequent interval is the interval of 0.6 to 0.7.

The processor 310 does not necessarily need to generate the histograms illustrated in FIGS. 4 and 5 in order to extract the most frequent interval in which the average values of the color component for the respective sub-regions are most frequently distributed among the predefined intervals. The histograms are included merely for convenience of explaining the function of the processor 310.

The processor 310 may extract an image composed only of the sub-regions corresponding to the extracted most frequent interval. Referring to FIG. 5, the processor 310 may perform step 550 of extracting only the sub-regions whose average V values fall within the most frequent interval (0.6 to 0.7).

Although the example in FIGS. 4 and 5 refers to the HSV color-coordinate system, any one of RGB, CIELab, YCbCr, Grayscale, or YUV may be used as the color-coordinate system. The processor 310 may, depending on the selected color coordinate system, use one of the color components (or color space elements) listed in Table 1 below, calculate an average value for each sub-region, and extract the most frequent interval in which the calculated average values are most frequently distributed.

TABLE 1

| Color Coordinate | Color component (or Color space |
|---|---|
| System | component) |
| RGB | R (Red), G (Green), B (Blue) |
| HSV | H (Hue), S (Saturation), V |
| | (Value) |
| CIELab | L (Lightness), a (degree of red |
| | (+) to green (−)), b (degree of |
| | yellow (+) to blue (−)) |
| YCbCr | Y (luminance/brightness), Cb |
| | (chrominance signal representing |
| | the difference between blue and |
| | luminance), Cr (chrominance |
| | signal representing the |
| | difference between red and |
| | luminance) |
| Grayscale | Brightness level of black and |
| | white |
| YUV | Y (luminance/brightness), U |
| | (blue luminance / blue |
| | chrominance), V (red luminance / |
| | red chrominance) |

Among the color coordinate systems listed in Table 1, in the case of the HSV color coordinate system, each color space component (or color component) is separated, and in particular, the V (Value, brightness) component is separated as an independent channel, thereby minimizing the influence of illumination changes. Because the color information is relatively less sensitive to lighting conditions compared to other color coordinate systems, more stable object recognition can be achieved. Therefore, it may be most preferable to use the HSV color coordinate system as the color coordinate system of the apparatus 300 for measuring biosignal based on the rPPG technology according to the present invention, and to use V (Value, brightness) as the color space component.

The processor 310 may calculate an average RGB value of the image including the sub-regions corresponding to the extracted most frequent interval. In addition, the processor 310 may output an RGB signal by arranging the average RGB values of the sub-regions composing the image in chronological order.

Referring to FIG. 5, after performing the step 550 of extracting the image for the sub-regions (or the image composed to the sub-regions) corresponding to the most frequent interval, the processor 310 may calculate average values of the RGB color coordinate system for the extracted sub-regions. When a plurality of images containing the user's face captured over time are provided as input, these average values of the RGB color coordinate system for the extracted sub-regions can serve as rPPG signals that can be readily used as input to various rPPG signal-processing devices or predetermined trained artificial intelligence models.

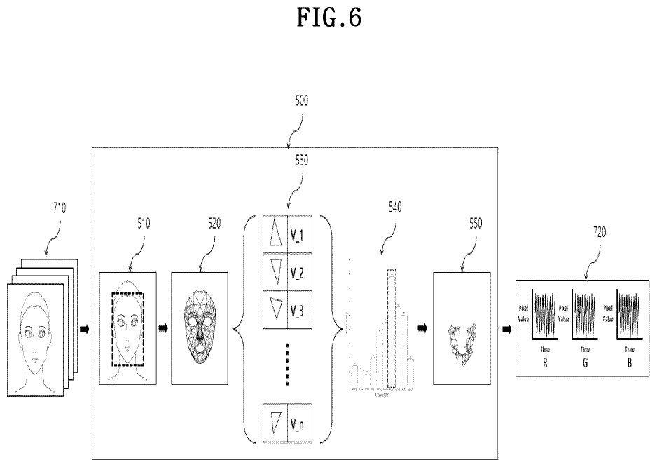

FIG. 6 is another exemplary diagram for explaining a part of the data-processing procedure of the apparatus 300 according to the present invention.

Referring to FIG. 6, additional processing steps corresponding to the preceding and subsequent stages of the data-processing procedure illustrated in FIG. 5 are depicted. The processor 310 may acquire (710) a plurality of images including the user's face obtained through continuously captured photographs or video by the camera, and, through the series of data-processing steps shown in FIG. 5, may ultimately output (720) RGB signals corresponding to variations or changes over time in R (Red), G (Green), and B (Blue) for the plurality of images.

When processing continuous images that include the same user's face within the same environment, a sudden change in the most frequent interval may result in degradation of the RGB (rPPG) signal quality. To address this issue, a correction algorithm may be considered for cases where the difference between the most frequent interval extracted from the immediately preceding image and the most frequent interval extracted from the current image is greater than a predetermined threshold.

In the above-described data-processing procedure, the processor 310 may extract the most frequent interval corresponding to the interval in which the average values of the color component calculated for the respective sub-regions are most frequently distributed, and may store the extracted intervals in a predetermined number. Here, the memory space for storing past most frequent intervals may preferably be implemented in a queue structure.

The processor 310 may compare the most frequent interval for the current image with the most frequent interval stored for the immediately preceding image, and when the difference exceeds the predetermined threshold, the processor 310 may determine the current image to be a noisy image. In such a case, instead of using the most frequent interval for the current image, the processor 310 may extract and use a median interval (or central interval) from the predetermined number of past most frequent intervals stored in the queue, and may refrain from storing the most frequent interval for the current image.

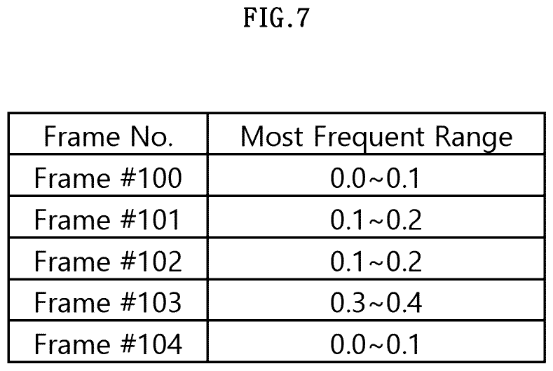

FIG. 7 is an exemplary diagram illustrating a process of extracting a central interval in the apparatus 300 according to the present invention.

Referring to FIG. 7, most frequent intervals corresponding to past image frames from the 100th frame to the 104th frame are stored. For example, when the threshold difference from the most frequent interval of the immediately preceding image is 0.3, and the most frequent interval for the current image frame (Frame #105) is greater than 0.9 and equal to or less than 1.0 (0.9 to 1.0), the most frequent interval for the previous image frame (Frame #104) is from 0.0 to 0.1 (0.0 to 0.1). The difference between the central values of these two intervals is 0.9 (0.95−0.05), which is greater than 0.3. Accordingly, the processor 310 may determine that the current image is a noisy image. In such a case, the processor 310 may extract, as the central interval, the past most frequent interval having the central value among the stored past intervals, such as the interval greater than 0.3 and equal to or less than 0.4 (0.3 to 0.4), and may extract an image including the sub-regions whose average color-component values correspond to the extracted central interval. In the specification, the image including the sub-regions may refer to an image that includes only the sub-regions.

When two intervals, rather than one, are extracted as central intervals from the stored past most frequent intervals, the processor 310 may calculate the central values of the two intervals and may extract, as the central interval, the interval that includes the average of the two calculated central values. For example, when the stored past most frequent intervals are 0.1 to 0.2, 0.2 to 0.3, 0.3 to 0.4, and 0.4 to 0.5, the central intervals are 0.2 to 0.3 and 0.3 to 0.4. In this case, the processor 310 may extract, as the central interval, the interval including the average of the two central values, i.e., 0.3 ((0.25+0.35)/2), which corresponds to the interval 0.2 to 0.3.

The processor 310 may calculate an average RGB value of the image for the sub-regions corresponding to the extracted central interval. In addition, the processor 310 may output an RGB signal by arranging the average RGB values of the sub-regions for the image (or composing image) in chronological order.

After extracting the image including the sub-regions corresponding to the central interval, the processor 310 may calculate average values of the RGB color-coordinate system for the extracted sub-regions. When a plurality of images containing the user's face, captured over time, are provided as input, the average RGB values for the extracted sub-regions may serve as rPPG signals that can be readily used as input to various rPPG signal-processing devices or predetermined trained artificial intelligence models.

The processor 310 may obtain a plurality of images including the user's face, obtained through continuously captured photographs or video by the camera, and, through a series of data-processing steps, may ultimately output RGB signals corresponding to variations or changes over time in R (Red), G (Green), and B (Blue) for the plurality of images.

The memory 330 may store all images including the user's face acquired from the camera 320, data related to face-region recognition and sub-region segmentation generated by the processor 310, algorithms for converting into predetermined color coordinate system, average values of color component (color space component), predefined intervals for color component or color space components most frequent intervals corresponding to intervals in which the average values of color component are most widely distributed, images including the sub-regions corresponding to the extracted most frequent interval; a predetermined number of recently extracted most frequent intervals, a central interval among the predetermined number of recently extracted most frequent intervals; images composed only of the sub-regions whose average color-component values correspond to the extracted central interval, and other data generated based thereon.

The apparatus and method for measuring a biosignal based on rPPG according to the present invention can effectively eliminate noise caused by the user's facial shape and surrounding illumination conditions. In particular, by performing region-specific analysis based on facial landmarks, the geometric characteristics and illumination-reflection properties of each region can be individually considered. This minimizes distortions caused by the curved structure or shading of the face and enables precise compensation for illumination variations on a region-by-region basis, thereby allowing more stable and accurate biosignal measurement.

The apparatus and method for measuring a biosignal based on rPPG according to the present invention improve upon conventional approaches that rely solely on simple skin-pixel analysis by dividing the facial region into finer units and independently evaluating the noise level of each region. Such an approach enables precise analysis based on the color composition of the color-coordinate system, allowing the system to more accurately identify and exclude skin regions unsuitable for biosignal measurement. In particular, subtle differences in skin tone and localized effects of specular or diffuse reflection can be accurately detected, thereby significantly enhancing measurement accuracy.

The apparatus and method for measuring a biosignal based on rPPG according to the present invention can dramatically improve the performance of biosignal measurement algorithms that use conventional rPPG measurement techniques. By combining facial-landmark-based segmentation and precise noise analysis using color-coordinate systems, more stable and accurate biosignal measurement becomes possible. This greatly enhances robustness against various environmental variables encountered in real-world usage conditions, thereby significantly improving the practicality and reliability of the algorithm.

The effects obtainable from the present invention are not limited to those described above, and other effects not explicitly mentioned will be clearly understood by those skilled in the art from the following description.

The apparatus described above may be implemented using hardware components, software components, and/or a combination of hardware and software components. For example, the apparatus and components described in the embodiments may be implemented using one or more general-purpose or special-purpose computers capable of executing and responding to instructions, such as a processor, controller, ALU (arithmetic logic unit), digital signal processor, microcomputer, FPGA (field-programmable gate array), PLU (programmable logic unit), microprocessor, or any other device capable of executing instructions. The processing device may execute an operating system (OS) and one or more software applications running on the operating system. In response to software execution, the processing device may access, store, manipulate, process, and generate data. For ease of understanding, certain descriptions herein refer to the processing device in the singular; however, those skilled in the art will appreciate that the processing device may include multiple processing elements and/or multiple types of processing elements. For example, the processing device may include multiple processors or a combination of a processor and a controller. Other processing configurations, such as a parallel processor, are also possible.

Software may include a computer program, code, instructions, or any combination thereof, and may configure or collectively instruct the processing device to operate in a desired manner. Software and/or data may be embodied permanently or temporarily on any type of machine, component, physical device, virtual apparatus, computer-readable storage medium, or signal wave, to allow interpretation by the processing device or to provide instructions or data thereto. Software may also be distributed across network-connected computing devices and stored or executed in a distributed manner. Software and data may be stored on non-transitory computer-readable storage medium or one or more computer-readable recording media.

Methods according to embodiments may be implemented as program instructions executable through various computer means and may be recorded on a computer-readable medium. The computer-readable medium may include program instructions, data files, data structures, or a combination thereof. The program instructions recorded on the medium may include those specially designed and configured for the embodiments or those known and available to those skilled in the art of computer software. Examples of computer-readable recording media include magnetic media such as hard disks, floppy disks, and magnetic tapes, optical media such as CD-ROMs and DVDs, magneto-optical media such as floptical disks; and hardware apparatuses specially configured to store and execute program instructions, such as ROM, RAM, and flash memory. Program instructions include machine-language code generated by a compiler as well as higher-level language code executable by a computer using an interpreter or the like. The hardware devices may be configured to operate as one or more software modules to perform the operations of the embodiments, and vice versa.

The embodiments described above represent combinations of components and features in predetermined forms. Each component or feature should be considered optional unless expressly stated otherwise. Each component or feature may be implemented without being combined with other components or features. Additionally, combinations of some components and/or features may constitute another embodiment of the invention. The order of operations described in the embodiments may be changed. Some components or features of one embodiment may be included in another embodiment or replaced with corresponding components or features of another embodiment. It is apparent that claims not explicitly recited as dependent in the claim set may nonetheless be combined to form various embodiments or may be incorporated as new claims through amendments after filing.

The processor 310 according to the present invention may be implemented by hardware, firmware, software, or a combination thereof. When implemented using hardware, the processor 310 may include ASICs (application-specific integrated circuits), DSPs (digital signal processors), DSPDs (digital signal processing devices), PLDs (programmable logic devices), FPGAs (field-programmable gate arrays), or other circuits configured to perform the operations of the invention. The present invention may also be embodied as a computer-readable recording medium storing a program for executing the method for preventing leakage of user information during user authentication.

The present invention may be embodied in other specific forms without departing from the essential characteristics of the invention, as will be apparent to those skilled in the art. Therefore, the foregoing detailed description is to be considered exemplary rather than limiting in all respects. The scope of the invention should be determined by the reasonable interpretation of the appended claims, and all modifications within the equivalent scope of the invention are to be included therein.

What is claimed is:

1. A apparatus for measuring a biosignal based on remote photoplethysmography (rPPG), comprising:
a camera configured to obtain an image including a face of a user; and
a processor operably coupled to the camera, and configured to:
recognize a facial region of the user from the obtained image and divide the facial region into a plurality of sub-regions according to a predetermined method;
convert each of the sub-regions into a predetermined color coordinate system and calculate, for each of the sub-regions, an average value of a color component included in the predetermined color coordinate system;
extract, among predefined intervals, a most frequent interval corresponding to an interval in which the average values of the color component calculated for the respective sub-regions are most frequently distributed;
when a difference between the extracted most frequent interval and a most frequent interval extracted immediately prior thereto is greater than or equal to a predetermined threshold, extract a central interval among a predetermined number of recently extracted most frequent intervals; and
extract an image including the sub-regions whose calculated average values of the color component correspond to the extracted central interval.

2. The apparatus of claim 1, wherein when the central interval among the predetermined number of recently extracted most frequent intervals comprises two intervals, the processor is further configured to calculate central values of the two intervals and extract, as the central interval, a single interval that includes an average of the calculated central values.

3. The apparatus of claim 1, wherein the processor is further configured to output an RGB signal corresponding to variations of R (Red), G (Green), and B (Blue) based on the image for the sub-regions corresponding to the most frequent interval.

4. The apparatus of claim 3, wherein the processor is configured to provide the output RGB signal to a Photoplethysmography (PPG) signal-processing device, an rPPG signal-processing device, or a trained artificial-intelligence model to measure various biosignals.

5. The apparatus of claim 1, wherein the processor is further configured to calculate an average RGB value of an image including the sub-regions whose calculated average values of the color component correspond to the extracted central interval.

6. The apparatus of claim 1, wherein the color coordinate system is any one of RGB, HSV, CIELab, YCbCr, Grayscale, or YUV.

7. The apparatus of claim 1, wherein the color component is:
any one of R (Red), G (Green), or B (Blue) when the color coordinate system is RGB;
any one of H (Hue), S (Saturation), or V (Value) when the color coordinate system is HSV;
any one of L (Lightness), a (a degree ranging from red(+) to green(−)), or b (a degree ranging from yellow(+) to blue(−)) when the color-coordinate system is CIELab;
any one of Y (luminance), Cb (blue-difference chroma signal), or Cr (red-difference chroma signal) when the color-coordinate system is YCbCr;
a grayscale brightness value when the color-coordinate system is Grayscale; and
any one of Y (luminance), U (blue luminance), or V (red luminance) when the color-coordinate system is YUV.

8. The apparatus of claim 1, wherein the processor is configured to recognize the facial region of the user from the obtained image and divide the facial region into a plurality of sub-regions by using a Mediapipe.

9. A method for measuring a biosignal based on remote photoplethysmography (rPPG), comprising:
obtaining an image including a face of a user;
recognizing a facial region of the user from the obtained image and dividing the facial region into a plurality of sub-regions according to a predetermined method;
converting each of the sub-regions into a predetermined color coordinate system and calculate, for each of the sub-regions, an average value of a color component included in the predetermined color coordinate system;
extracting, among predefined intervals, a most frequent interval corresponding to an interval in which the average values of the color component calculated for the respective sub-regions are most frequently distributed;
when a difference between the extracted most frequent interval and a most frequent interval extracted immediately prior thereto is greater than or equal to a predetermined threshold, extracting a central interval among a predetermined number of recently extracted most frequent intervals; and
extracting an image including the sub-regions whose calculated average values of the color component correspond to the extracted central interval.

10. The method of claim 9, further comprising:
wherein when the central interval among the predetermined number of recently extracted most frequent intervals comprises two intervals, calculating central values of the two intervals and extract, as the central interval, a single interval that includes an average of the calculated central values.

11. The method of claim 9, further comprising;
calculating an average RGB value of an image including the sub-regions whose calculated average values of the color component correspond to the extracted central interval.

12. A non-transitory computer-readable storage medium storing instructions that, when executed by one or more processors, cause the one or more processors to:
obtain an image including a face of a user;
recognize a facial region of the user from the obtained image and divide the facial region into a plurality of sub-regions according to a predetermined method;
convert each of the sub-regions into a predetermined color coordinate system and calculate, for each of the sub-regions, an average value of a color component included in the predetermined color coordinate system;
extract, among predefined intervals, a most frequent interval corresponding to an interval in which the average values of the color component calculated for the respective sub-regions are most frequently distributed;
when a difference between the extracted most frequent interval and a most frequent interval extracted immediately prior thereto is greater than or equal to a predetermined threshold, extracting a central interval among a predetermined number of recently extracted most frequent intervals; and
extract an image including the sub-regions whose calculated average values of the color component correspond to the extracted central interval.
